(19) United States
(12) Patent Application Publication
(10) Pub. No.: US 2020/0054285 A1
(43) Pub. Date: Feb. 20, 2020

(54) Electrode Patch

(71) Applicant: Brain Sentinel, Inc., San Antonio, CA (US)

(72) Inventors: Eliza Lemons, San Antonio, TX (US); Luke E. Whitmire, San Antonio, TX (US)

(21) Appl. No.: 16/346,379

(22) PCT Filed: Oct. 31, 2017
(86) PCT No.: PCT/US2017/059382

§ 371 Date: Apr. 30, 2019

Related U.S. Application Data
(60) Provisional application No. 62/415,322, filed on Oct. 31, 2016.

(51) Int. Cl. A61B 5/00

(52) U.S. Cl. CPC A61B 5/68335; A61B 2562/164; A61B 5/6824; A61B 5/6833

(57) ABSTRACT

An electrode patch comprising a lobed patch body and sensor studs having hydrogel caps disposed therein.

### CROSS-REFERENCE TO RELATED APPLICATION

This application claims priority to U.S. Provisional Application No. 62/415,322 entitled “Electrode Patch” filed Oct. 31, 2016, the disclosure of which is hereby entirely incorporated herein by reference.

### FIELD

The disclosed method and apparatus generally relate to sensor electrodes for detecting physiological phenomena.

### SUMMARY

An electrode patch comprising a patch body having a skin side and a device side, the skin side having an adhesive coating; an electrode base coupled to the patch body; a plurality of electrode studs securely mounted to the electrode body, the electrode studs each being configured for electrical connection to a sensor device at the device side of the patch body; and a plurality of hydrogel caps securely mounted to the electrode studs, each of the plurality of hydrogel caps being in electrical contact with one of the plurality of electrode studs.

### BRIEF DESCRIPTION OF THE DRAWINGS

FIG. 1 illustrates an exploded view of one embodiment of an electrode patch.

FIG. 2 illustrates a skin-side view of the embodiment of FIG. 1 having a release liner partially removed.

FIG. 3 illustrates a skin-side view of the embodiment of FIG. 1 having a release liner fully removed.

FIG. 4 illustrates a device-side view of the embodiment of FIG. 1 having a release liner.

FIG. 5 illustrates a device-side view of the embodiment of FIG. 1 having a release liner fully removed.

FIG. 6 illustrates a perspective view of an example sensor device configured to connect with the patch embodiment of FIG. 1.

FIG. 7 illustrates a bottom view of the embodiment of FIG. 6.

FIG. 8 illustrates one example of placement of an electrode patch on a human body.

FIG. 9 illustrates a sensor device attached to an electrode patch.

FIG. 10 illustrates a sensor device attached to an electrode patch placed on a human body.

FIG. 11 is a top perspective view of another embodiment of an electrode patch.

FIG. 12 is a rear elevational view of the embodiment of FIG. 11.

FIG. 13 is a front elevational view of the embodiment of FIG. 11.

FIG. 14 is a left side view of the embodiment of FIG. 11.

FIG. 15 is a right side view of the embodiment of FIG. 11.

FIG. 16 is a top plan view of the embodiment of FIG. 11.

FIG. 17 is a bottom plan view of the embodiment of FIG. 11.

FIG. 18 is a bottom perspective view of the embodiment of FIG. 11.

FIG. 19 is a top perspective view of yet another embodiment of an electrode patch.

FIG. 20 is a rear elevational view of the embodiment in FIG. 19.

FIG. 21 is a front elevational view of the embodiment in FIG. 19.

FIG. 22 is a left side elevational view of the embodiment in FIG. 19.

FIG. 23 is a right side elevational view of the embodiment in FIG. 19.

FIG. 24 is a top plan elevational view of the embodiment in FIG. 19.

FIG. 25 is a bottom plan elevation view of the embodiment in FIG. 19.

FIG. 26 is a bottom perspective view of the embodiment in FIG. 19.

FIG. 27 is a top perspective view of another embodiment of an electrode patch.

FIG. 28 is a rear elevational view of the embodiment in FIG. 27.

FIG. 29 is a front elevational view of the embodiment in FIG. 27.

FIG. 30 is a left side elevational view of the embodiment in FIG. 27.

FIG. 31 is a right side elevational view of the embodiment in FIG. 27.

FIG. 32 is a top plan view of the embodiment in FIG. 27.

FIG. 33 is a bottom plan view of the embodiment in FIG. 27.

FIG. 34 is a bottom perspective view of the embodiment in FIG. 27.

FIG. 35 is a top perspective view of yet another embodiment of an electrode patch.

FIG. 36 is rear elevational view of the embodiment in FIG. 35.

FIG. 37 is a front elevational view of the embodiment in FIG. 35.

FIG. 38 is a left side elevational view of the embodiment in FIG. 35.

FIG. 39 is a right side elevational view of the embodiment in FIG. 35.

FIG. 40 is a top plan view of the embodiment in FIG. 35.

FIG. 41 is a bottom plan view of the embodiment in FIG. 35.

FIG. 42 is a bottom perspective view of the embodiment in FIG. 35.

### DETAILED DESCRIPTION

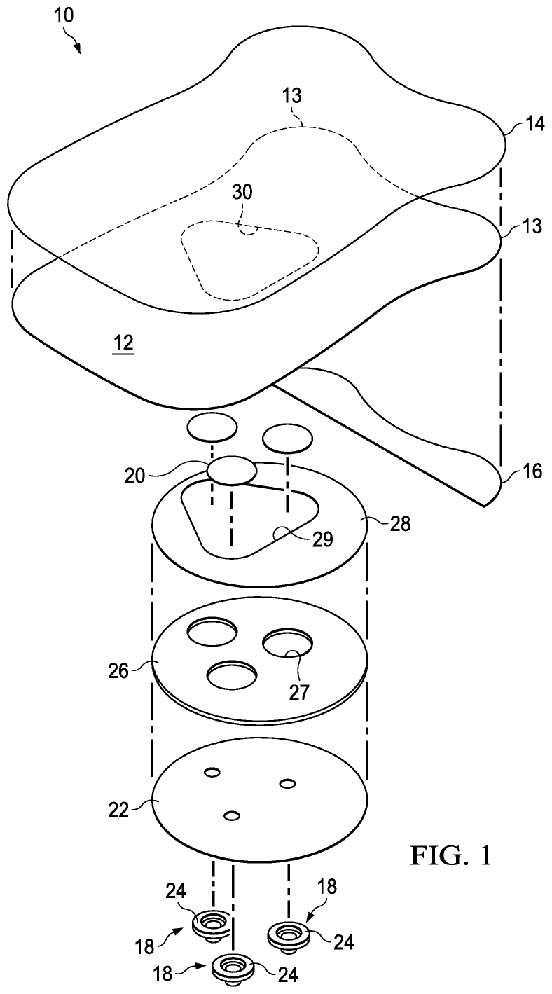

FIG. 1 illustrates an embodiment of an electrode patch 10 that may be used to detect physiological phenomena, and particularly electromyography signals generated by the human body. The electrode patch 10 comprises a patch body 12. The patch body 12 may comprise a flexible material having a pressure-sensitive adhesive coating, such as an acrylate adhesive, on one side. The adhesive coating may permit the patch to removably adhere to the skin. The flexible material may comprise a woven material, a plastic film or fabric mesh. The flexible material may comprise a breathable yet waterproof or water-resistant material, such as Gore-Tex™ fabric membrane. An adhesive release liner 14 may be disposed over the adhesive to protect the adhesive prior to use on the skin. The release liner 14 may comprise any material that may be readily removed from the adhesive without substantially reducing the binding capability of the adhesive.

The electrode patch 10 may comprise a plurality of lobes 13 that provide additional shear resistance when a sensor device is attached to the electrode patch 10 as described below.

A release tab 16 may be permanently affixed to the non-skin side of the patch body 12. The release tab 16 may comprise a material stiffer than the patch body 12 so as to permit the patch body 12 to be more easily grasped and removed from the skin.

The electrode patch 10 further comprises a plurality of electrodes. Each electrode comprises a sensor stud 18 and a hydrogel cap 20. The sensor stud 18 may comprise any suitable electrically-conductive material, such as Ag/AgCl. A hydrogel cap 20 may be disposed partially within and extending from the sensor stud 18. The hydrogel cap 20 may be provided for more robust electrode-skin interface, and may comprise a gel of sufficient elasticity to deform for patch use without permanently changing shape. In other embodiments, the hydrogel cap 20 may permanently change shape upon application to the skin, but may be sufficiently viscous that it remains coherent and substantially cap-shaped upon removal from the skin. The hydrogel cap 20 may thus leave little or no residue on the skin. Use of a hydrogel cap 20 may advantageously avoid the need for application of a conductive jelly prior to application of the patch. Use of a hydrogel cap 20 may also advantageously avoid messiness and reduce user error when applying the patch to skin. By connecting each hydrogel cap 20 to a sensor stud 18, the hydrogel cap 20 is better prevented from spreading too far from the sensor stud 18, thus substantially reducing the likelihood of an electrode short circuit in which two electrodes are electrically connected through hydrogel at the surface of the skin.

The sensor studs 18 may be permanently affixed to one side of an electrode base 22. In some embodiments, the sensor studs 18 may be affixed to the electrode base 22 using an adhesive 24. In other embodiments, the sensor studs 18 may be formed as part of the electrode base 22. For example, the electrode base 22 may comprise a generally non-conductive material (such as plastic, wood, ceramic or glass), and the sensor studs 18 may be coated with an electrically-conductive material. The hydrogel caps 20 may be coupled to the sensor studs 18 through the electrode base 22. The electrode base 22 may be used to maintain a particular spacing and configuration for the electrodes that may otherwise be difficult to maintain using only a flexible material patch body 12.

A spacer 26 may be used to accommodate the thickness of the hydrogel caps 20. The electrode base 22 may be affixed to the spacer 26 by adhesive. The spacer 26 may comprise any suitable material, such as a closed-cell white foam. The hydrogel caps 20 may be disposed in one or more openings 27 in the spacer 26. The spacer 26 may be affixed to the patch body 10 by an adhesive layer 28, or by any other suitable means, such as RF weld or hook-and-loop fastener. In some embodiments, the spacer 26 and electrode base 22 may comprise a unitary item of manufacture. For example, the spacer 26 and electrode base 22 may comprise the same structure. The sensor studs 18 may comprise a wide base, such as a metal disc, that allow the sensor studs 18 to better attach to the electrode base 22 by sandwiching or attaching the electrode base 22 between or to more electrode surface area. A wide base may further comprise a thickness suitable for preventing the sensor stud 18 from penetrating too deeply into the receiving port of a sensor device (see FIG. 7). By reducing such penetration, the force required to disengage the sensor device from the electrode patch may be reduced. A wide base may also provide a greater electrically-conductive surface area for attachment of a hydrogel cap 20. In some embodiments, the spacer 26 may be compressed by the electrode base 22, thus forming a shallow well or depression in which a hydrogel cap 20 may be disposed. In some embodiments, the hydrogel cap 20 stays embedded in the electrode so as to not be left behind when removing the electrode patch 10 from the skin. Without embedding the hydrogel cap 20 in the sensor stud 18, the hydrogel may be left behind on the human skin when a patch is removed for replacement by a new patch. The remaining hydrogel could bridge two electrodes of a newly applied electrode patch, thus potentially shorting out the electrodes of the newly applied patch. By joining the hydrogel cap 20 to the sensor stud 18, the risk of electrically connecting two electrodes at the surface of the skin may be advantageously reduced.

In the embodiment of FIG. 1, the adhesive layer 28 forms a void 29 through which the hydrogel caps 20 may extend. The patch body 12 similarly forms a void 30 through which the hydrogel caps 20 may extend for contact with the skin.

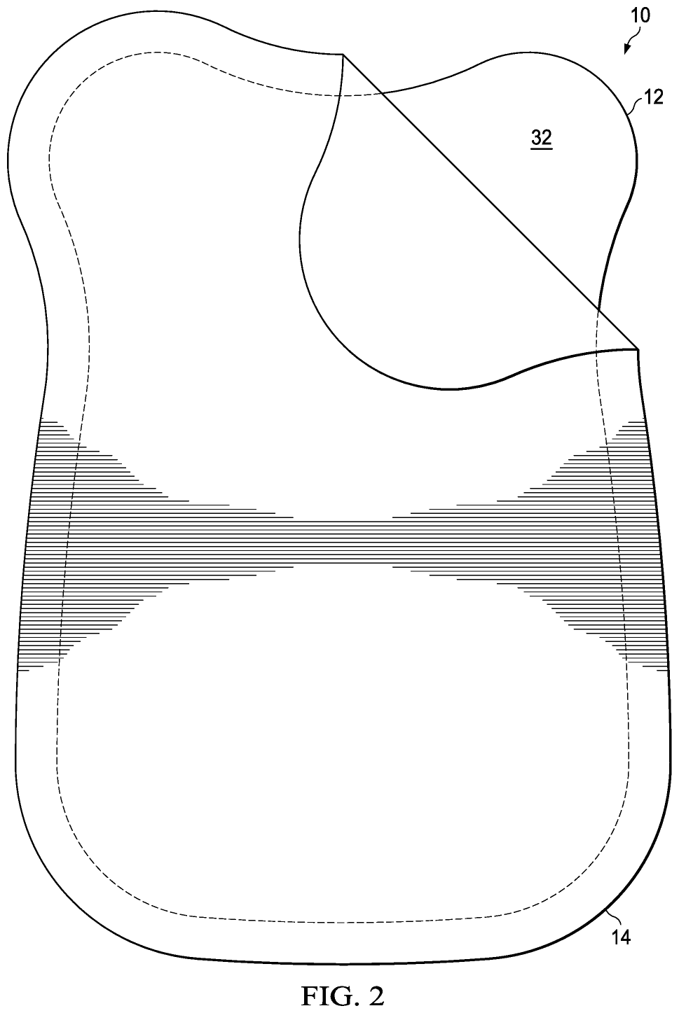

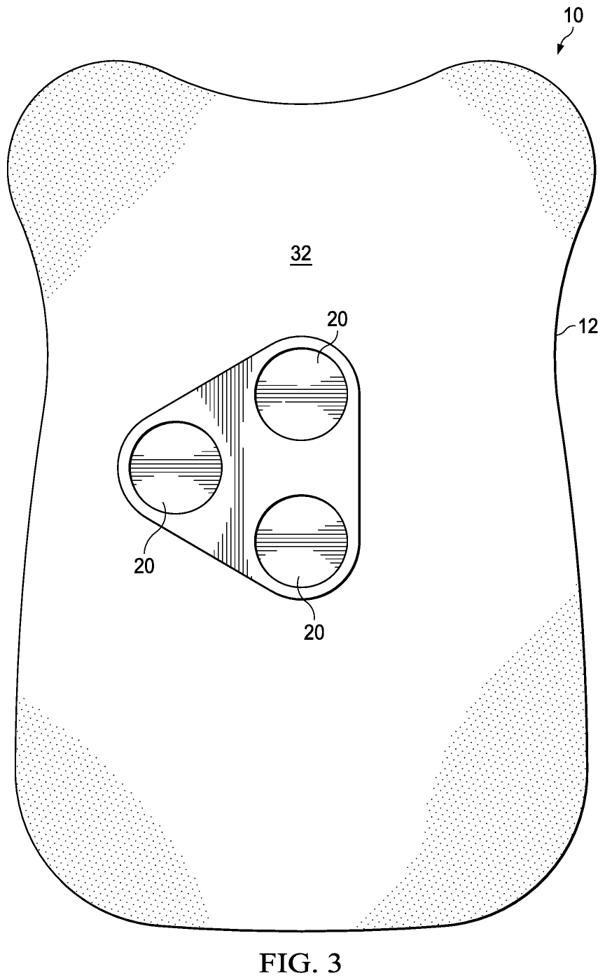

FIG. 2 illustrates a skin-side view of the electrode patch 10 having the release liner 14 partially removed from the patch body 12 to expose the adhesive 32. FIG. 3 illustrates a skin-side view of the electrode patch 10 having the release liner 14 fully removed from the patch body 12 to expose the adhesive 32. The void 30 in the patch body 12 exposes the hydrogel caps 20 for contact with the skin.

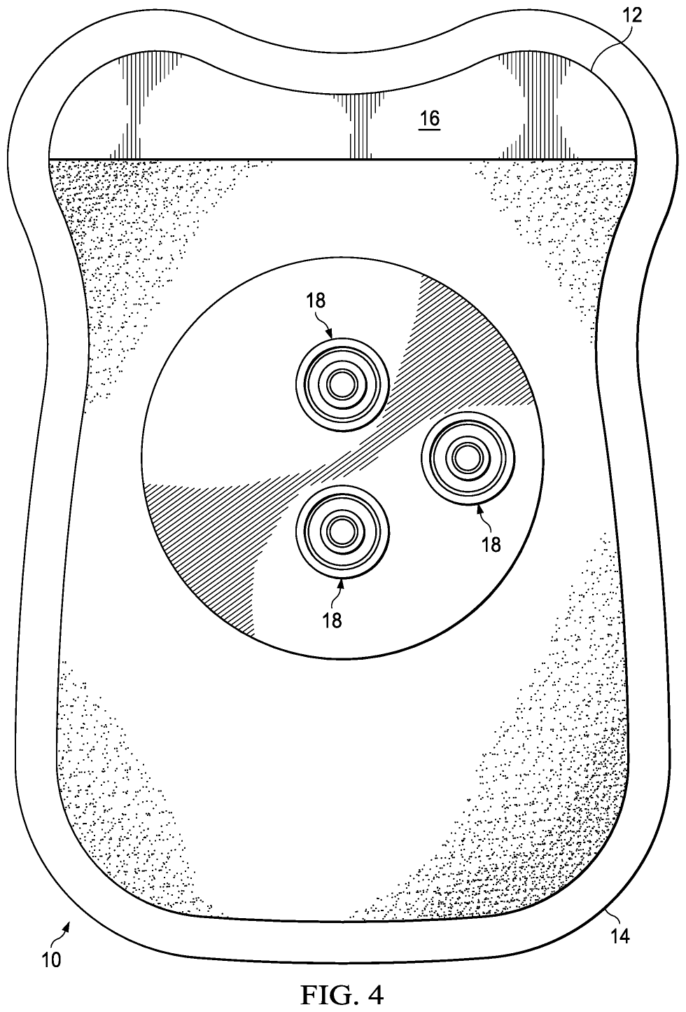

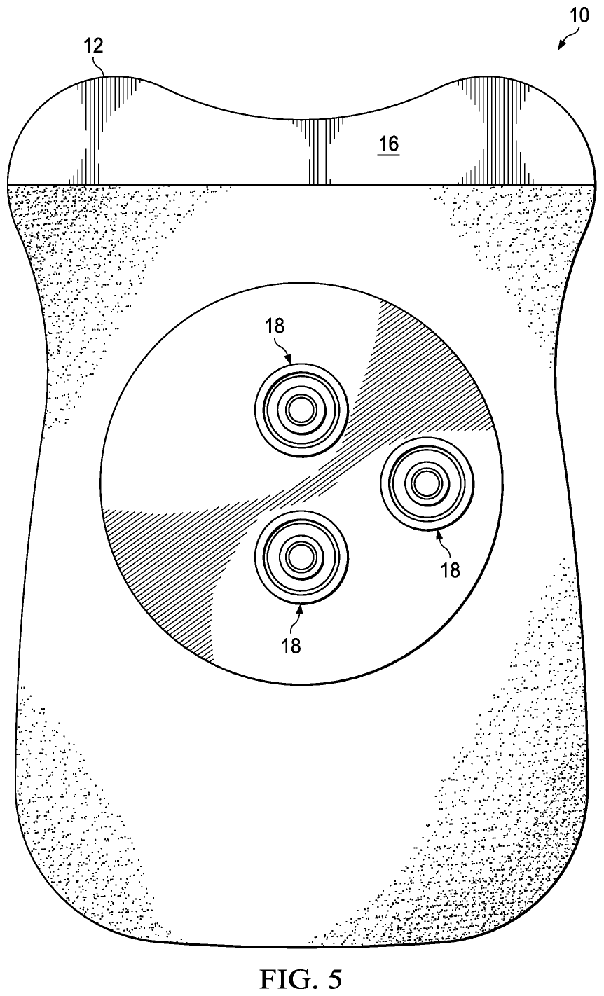

FIG. 4 illustrates a device-side view of the electrode patch 10 having the release liner 14 still covering the adhesive side of the patch body 12. The release tab 16 permits easier manipulation of the electrode patch 10. The sensor studs 18 may be exposed for contact with a sensor device. FIG. 5 illustrates a device-side view of the electrode patch 10 having the release liner 14 removed from the adhesive side of the patch body 12.

FIG. 6 illustrates an embodiment of a sensor device 40 configured to mount to the electrode patch. The sensor device includes a plurality of stud ports 42 configured to receive sensor studs of the electrode patch. FIG. 7 illustrates a patch-side view of the sensor device 40.

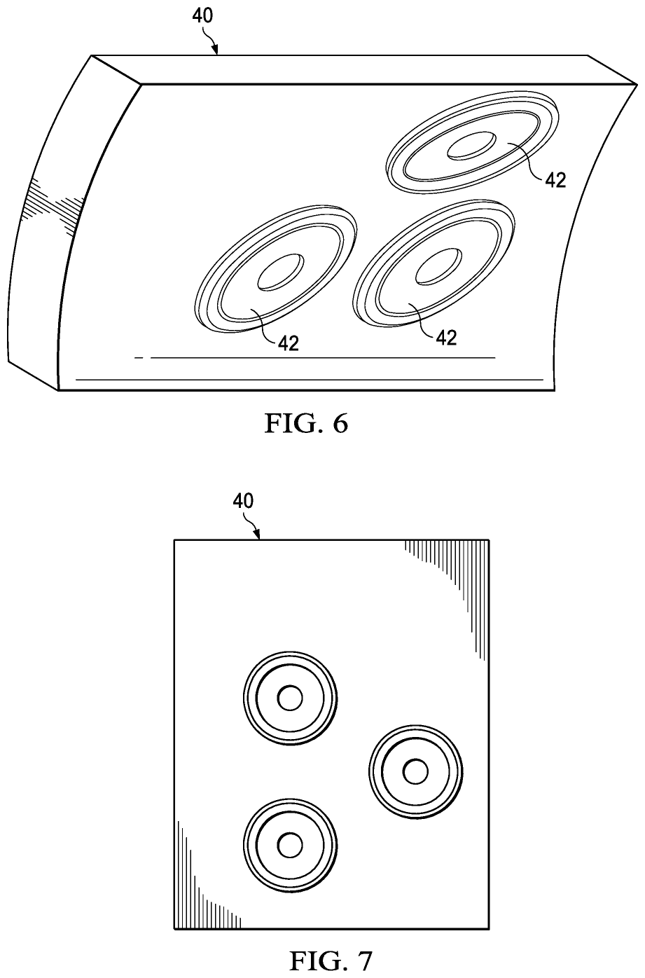

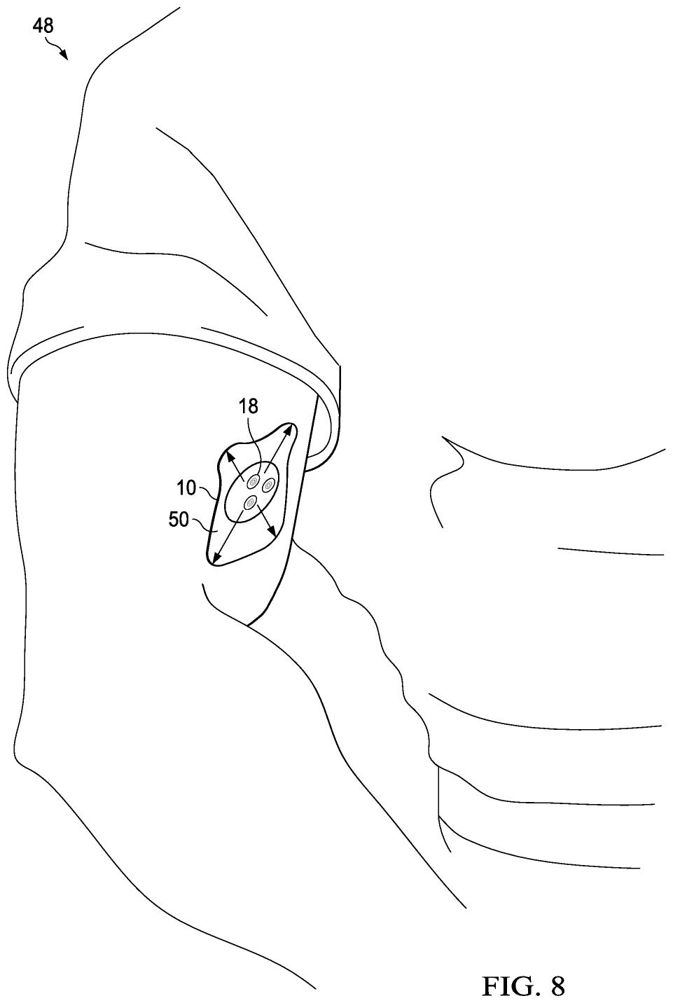

FIG. 8 illustrates an example of placement of the electrode patch 10 on the human body 48. The release liner may be removed from the skin side of the patch, and the electrode patch 10 may be adhered to the skin so that the hydrogel caps contact the skin. The sensor studs 18 may be exposed on device side 50 of the patch 10 and available for electrical connection to a sensor device (not shown). As may be seen in FIG. 9, a sensor device 40 may be connected to the electrode patch 10.

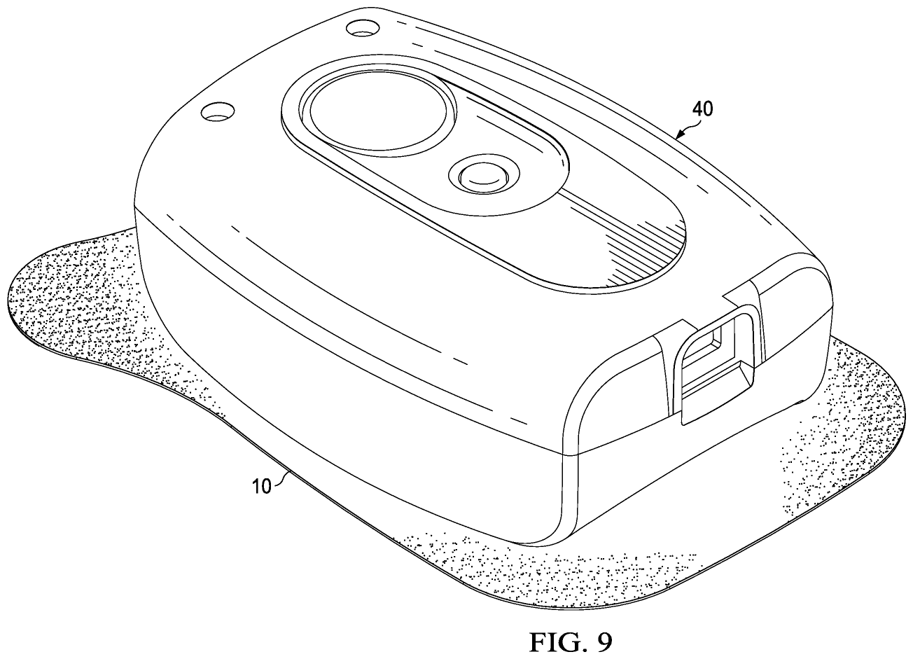

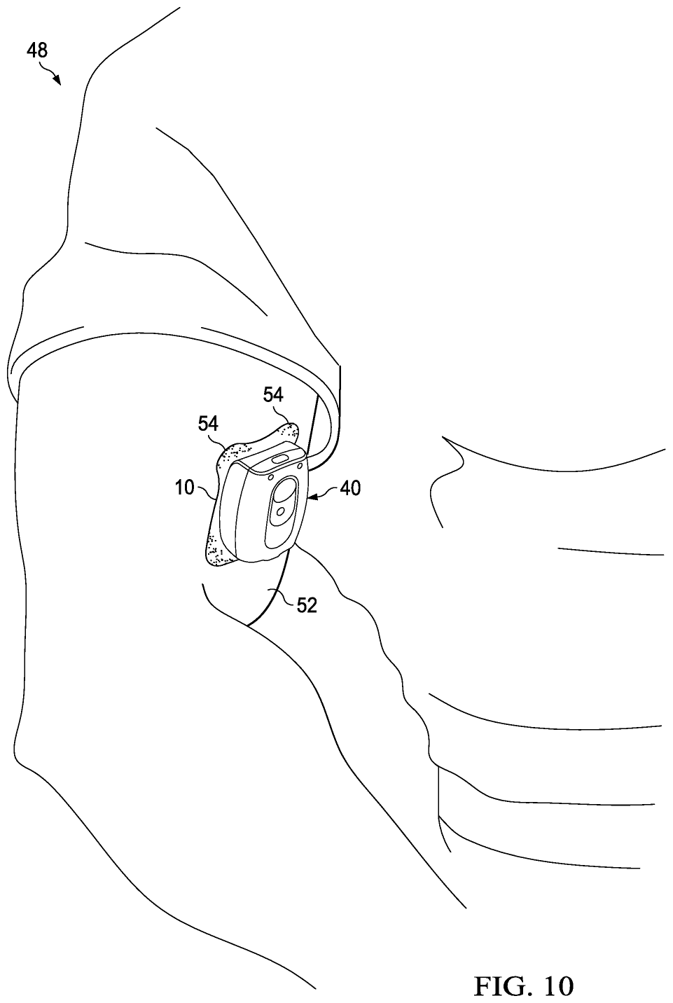

As may be seen in FIG. 10, the electrode patch 10 may be removably attached to the human body 48, such as over the biceps muscle 52. In the embodiment of FIG. 10, the electrode patch 10 may be oriented so that the lobes 54 may more effectively bear the weight of the sensor device 40. In the embodiment of FIGS. 8 and 10, the sensor device hangs from the electrode patch 10, thus providing shear forces between the electrode patch 10 and the wearer's skin. The lobes 54 may better maintain contact of the electrode patch 10 with the skin and reduce skin irritation by advantageously spreading the weight of the sensor device 40 across more patch surface area, and by increasing the length of the patch diagonals (shown in FIG. 8). Such an orientation may allow better patch adhesion through a wider arm movements and forces. Furthermore, electrode patches made of a flexible material, such as fabric, mesh or plastic, may better allow the skin to stretch and flex as the human body moves while still substantially maintaining electrode spacing.

In some embodiments, two types of material may be used to distribute the weight of a sensor device. A more cloth-like material may be used for the patch body to move with the patient's skin, and a more rigid electrode base may be used to provide sufficient firmness and structure to keep the electrodes spaced appropriately. Using an electrode base alone without using a patch body of flexible material may tend to exacerbate shear forces around the outside of the electrode base that may undesirably irritate the skin, such as by leaving a red mark on the skin. By adding a flexible patch body, the shear forces may be spread over a larger area. The shape of the patch body may be configured to increase the effective surface area to reduce shear forces between the electrode patch and the skin tissue to which it is attached, and use of lobes in the direction of primary shear forces may allow the benefits of force distribution with a smaller patch body. Use of lobes may thus further reduce the skin area susceptible to patch irritation, yet maintain sufficient patch-skin contact along major shear force vectors that may be generated during typical body movement.

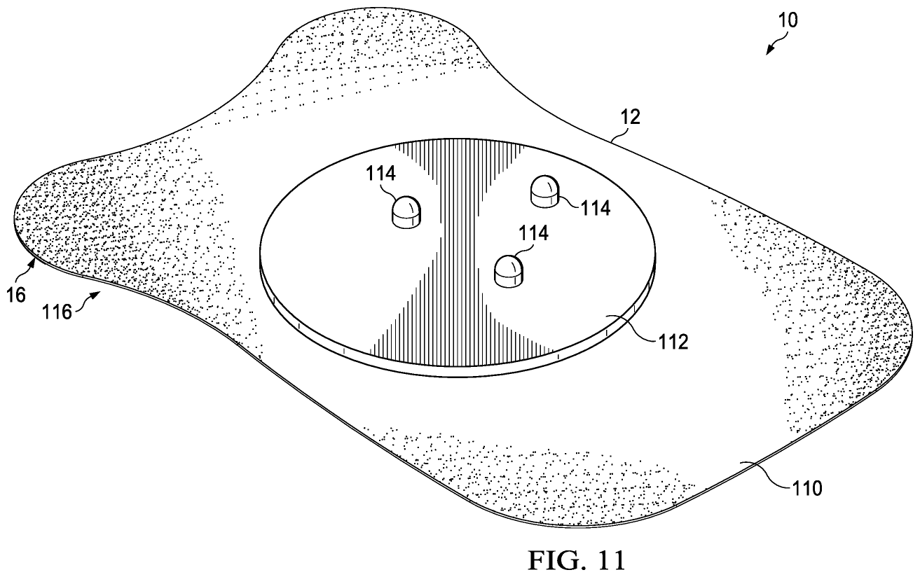

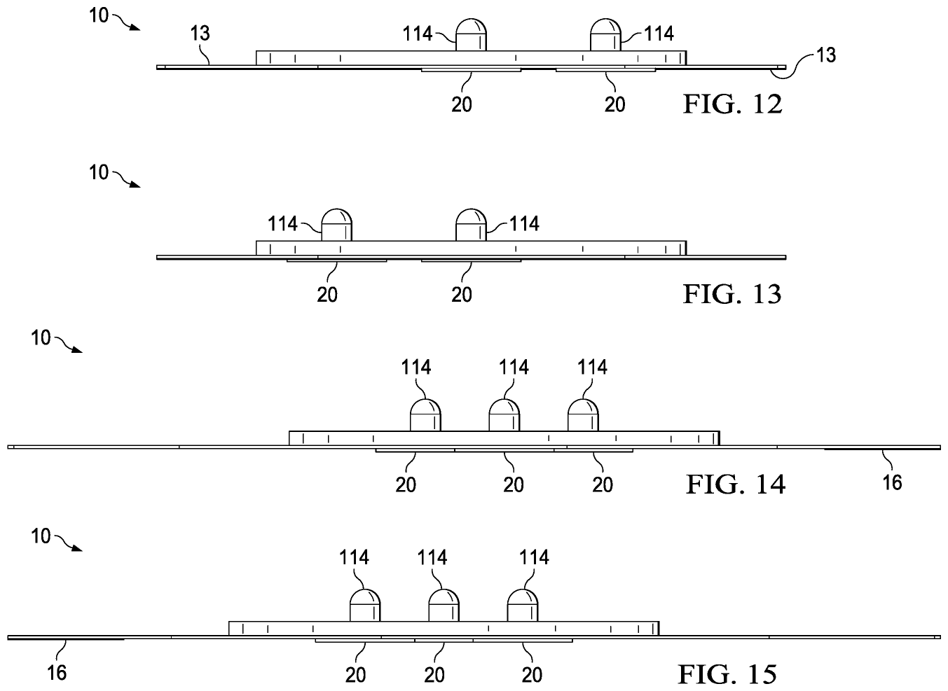

FIG. 11 illustrates a top perspective view of an embodiment of an electrode patch 10 having the components thereof assembled and the release liner 14 removed. The device side 110 of the electrode patch 10 may have an electrode assembly 112 permanently attached to the patch body 12. Electrode studs 114 may be exposed so as to permit connection of a sensor device (not shown) to the electrode patch 10. In the embodiment of FIG. 11, a tab 16 may be attached to the skin side 116 of the electrode patch 10. The tab 16 may be removably or permanently attached to the patch body. An adhesive coats the skin side of the electrode patch 10 so as to permit the electrode patch 10 to be removably mounted to the human body (not shown). FIGS. 12-15 respectively illustrate the four sides of the electrode patch 10 such that the electrode studs 114 may be seen as protruding from the device side of the electrode patch 10, and the hydrogel caps 20 may be seen as extending slightly beyond the skin-side surface of the electrode patch 10 so as to better contact skin. FIG. 13 illustrates a front elevational view showing the lobes 13 of the electrode patch. FIG. 12 illustrates a rear side view. FIG. 14 illustrates a left side view, and FIG. 15 illustrates a right side view.

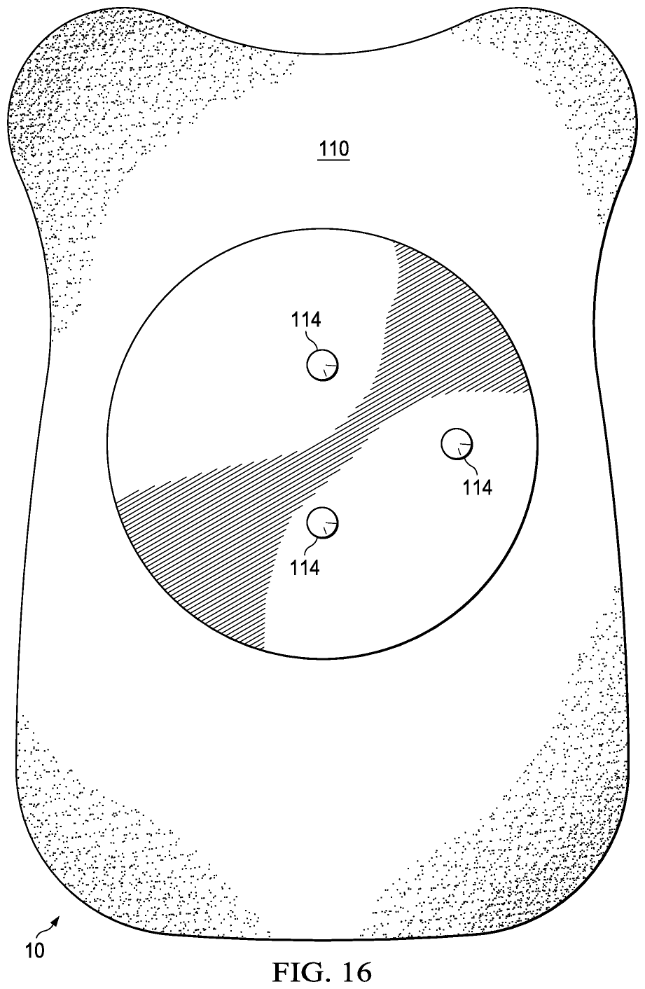

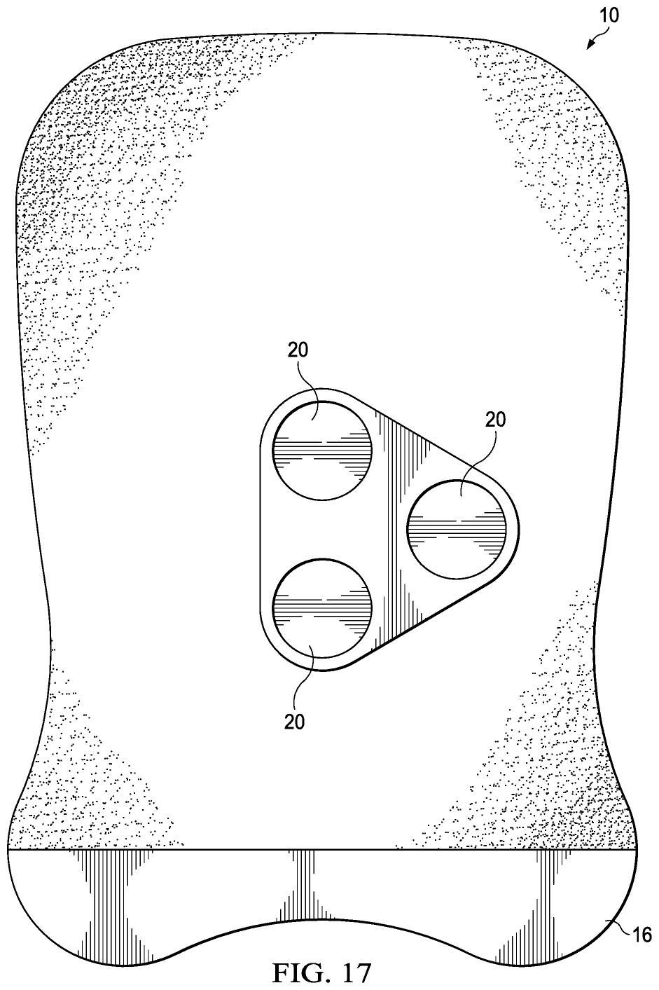

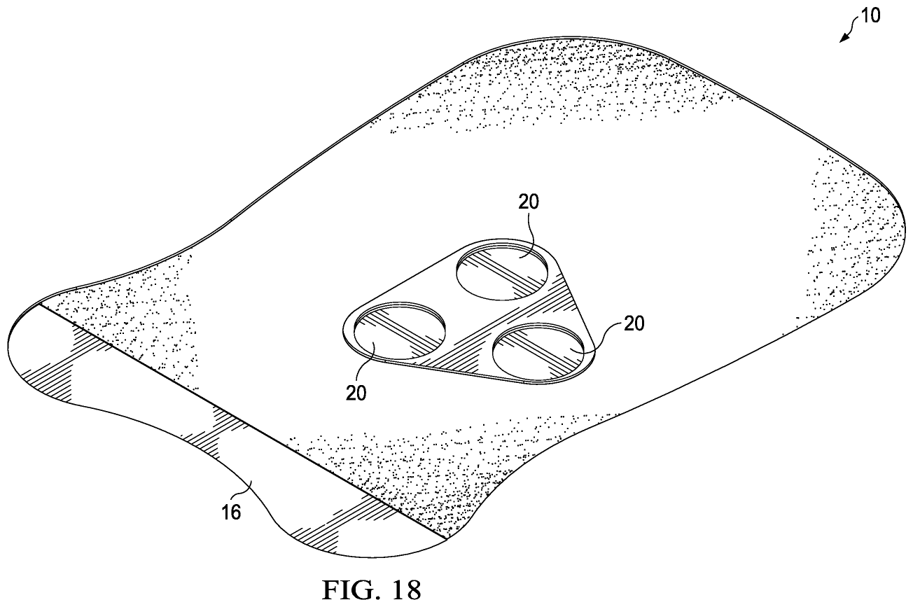

FIGS. 16-18 further illustrates a top plan view, a bottom plan view and a bottom perspective view, respectively, of the embodiment of FIG. 11.

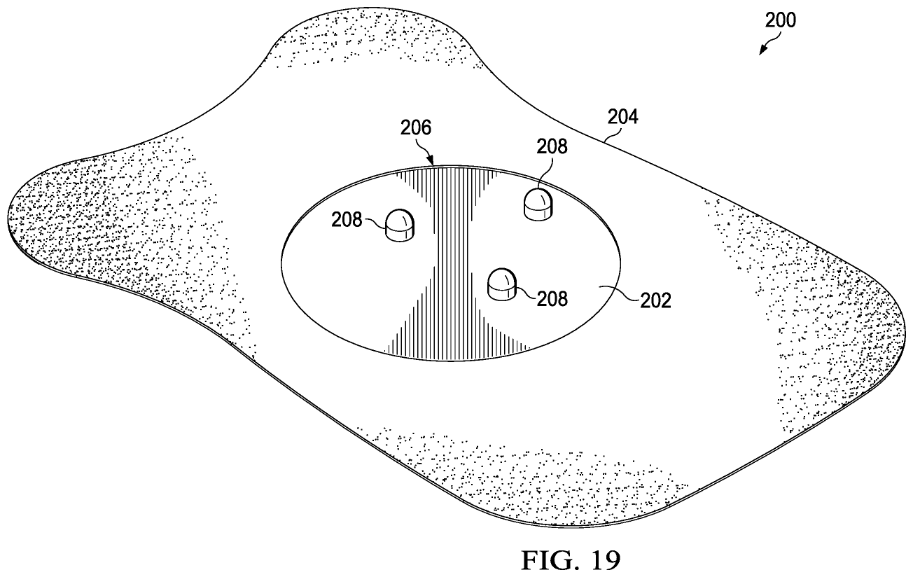

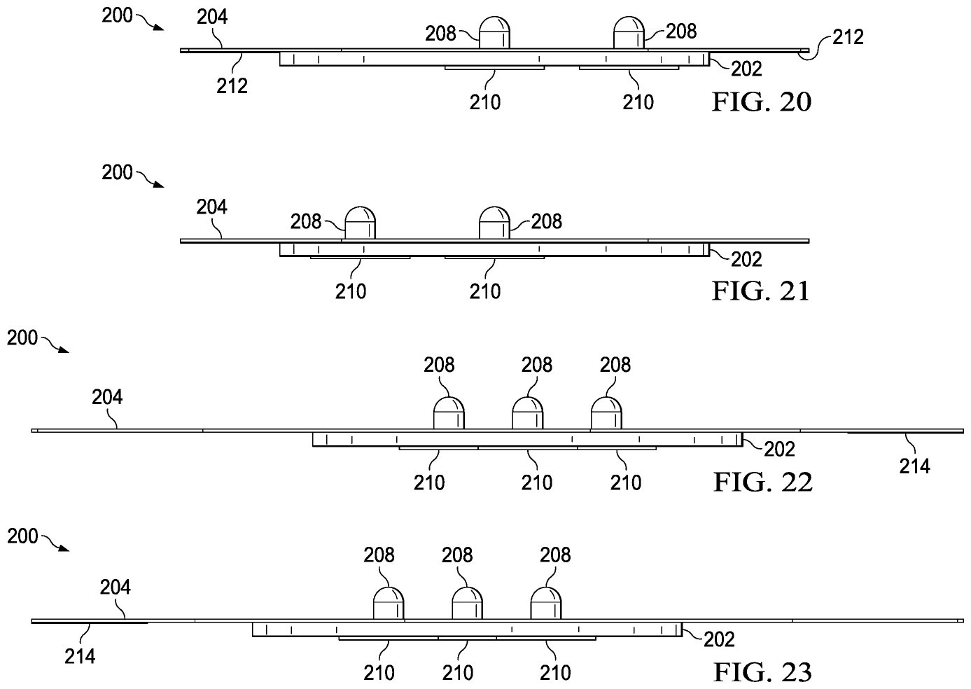

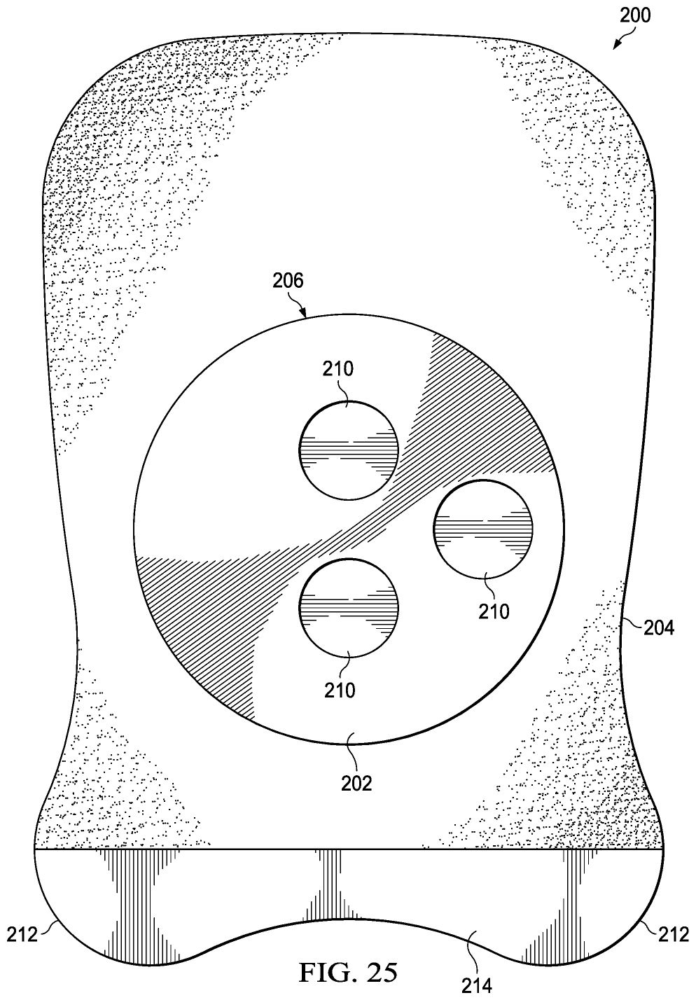

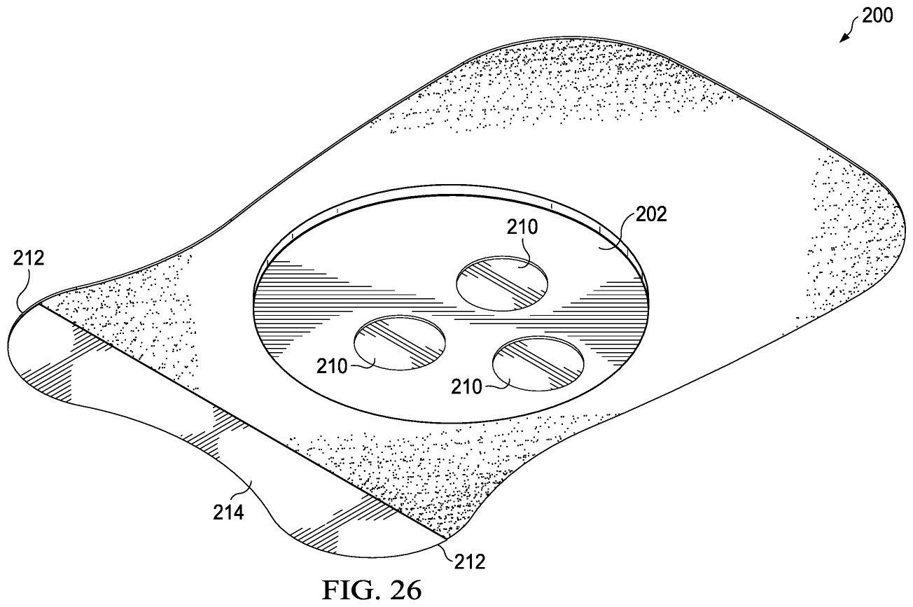

FIGS. 19-26 illustrate an embodiment of an electrode patch 200 in which the electrode base/spacer 202 may be disposed on the skin side of the patch body 204. As may be seen in the top perspective view of FIG. 19, the patch body 204 may form a void 206 through which electrode studs 208 may extend for connection to a sensor device (not shown). FIGS. 20-23 respectively illustrate the four sides of the electrode patch 200 such that the electrode studs 208 may be seen as protruding from the device side of the electrode patch 200, and the hydrogel caps 210 may be seen as extending slightly beyond the skin-side surface of the electrode patch 10 so as to better contact skin. FIG. 21 illustrates a front elevational view showing the lobes 212 of the electrode patch. FIG. 20 illustrates a rear side view. FIG. 22 illustrates a left side view, and FIG. 23 illustrates a right-side view.

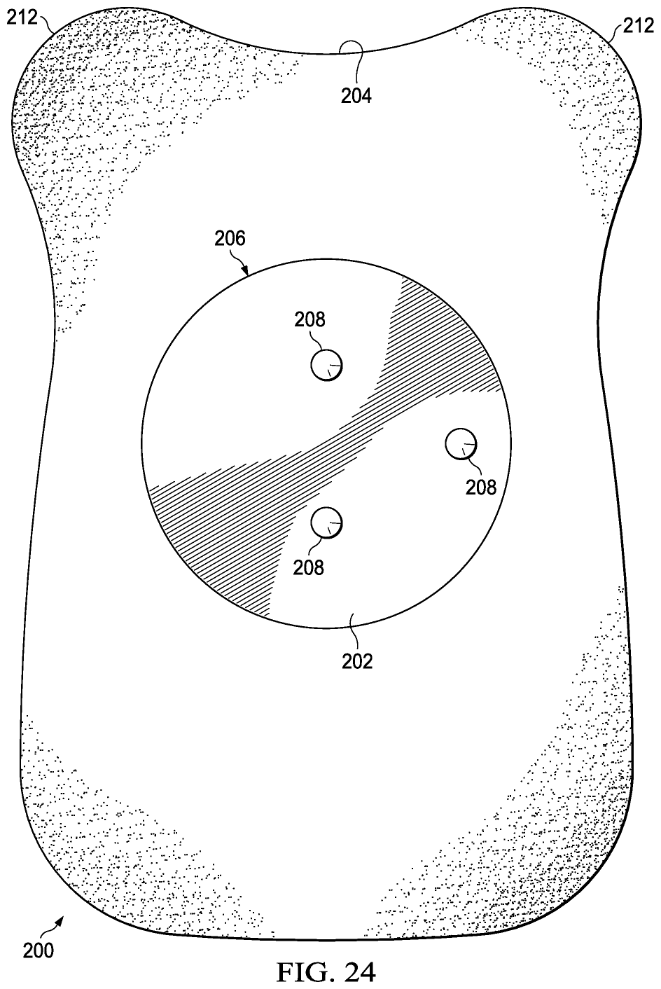

FIGS. 24-26 further illustrates a top plan view, a bottom plan view and a bottom perspective view, respectively, of the embodiment of FIG. 19. In the embodiment of FIG. 19, a tab 214 may be permanently or removably disposed on the skin side of the electrode patch 200. Alternatively, the tab 214 may be permanently or removably disposed on the device side of the electrode patch 200.

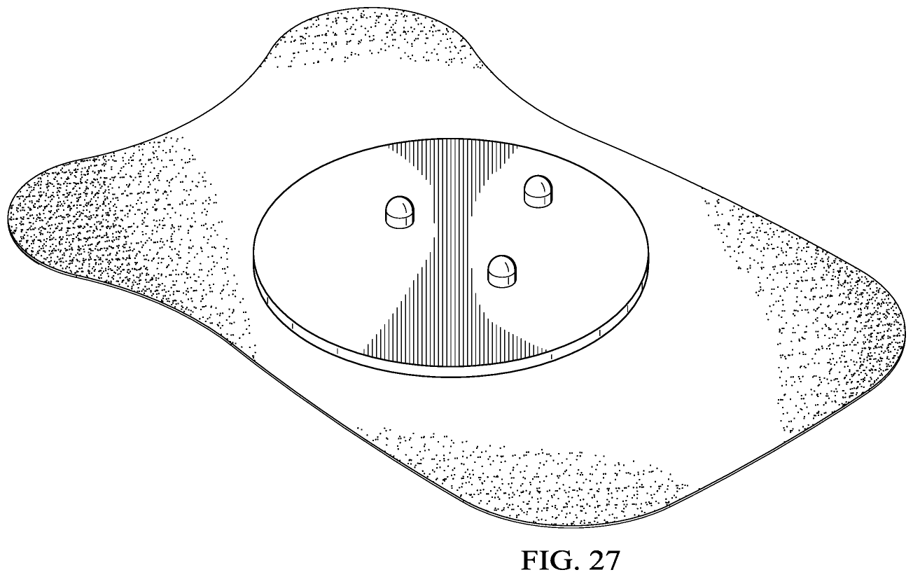

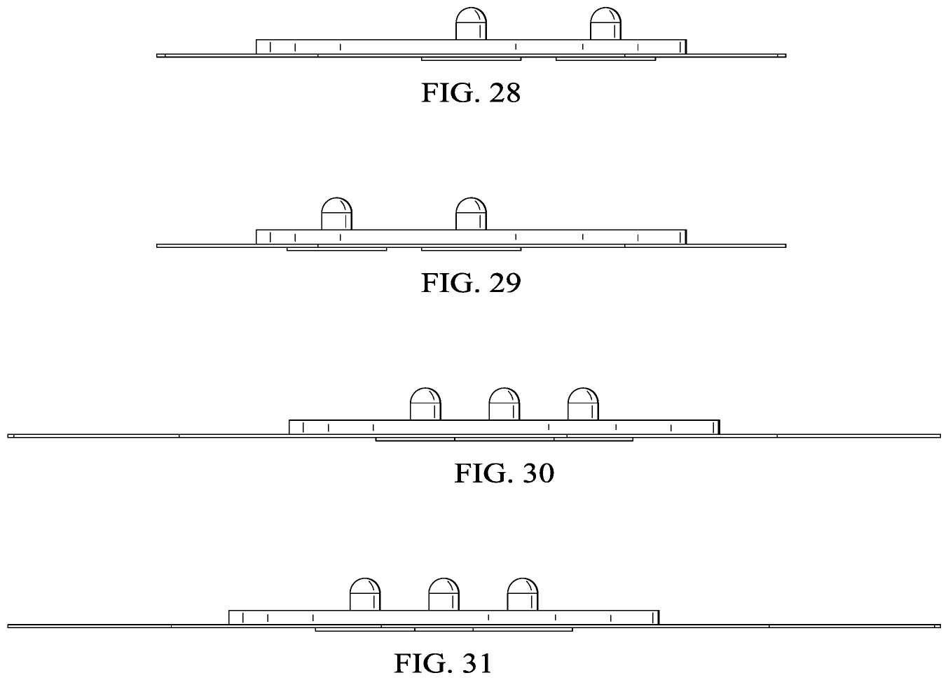

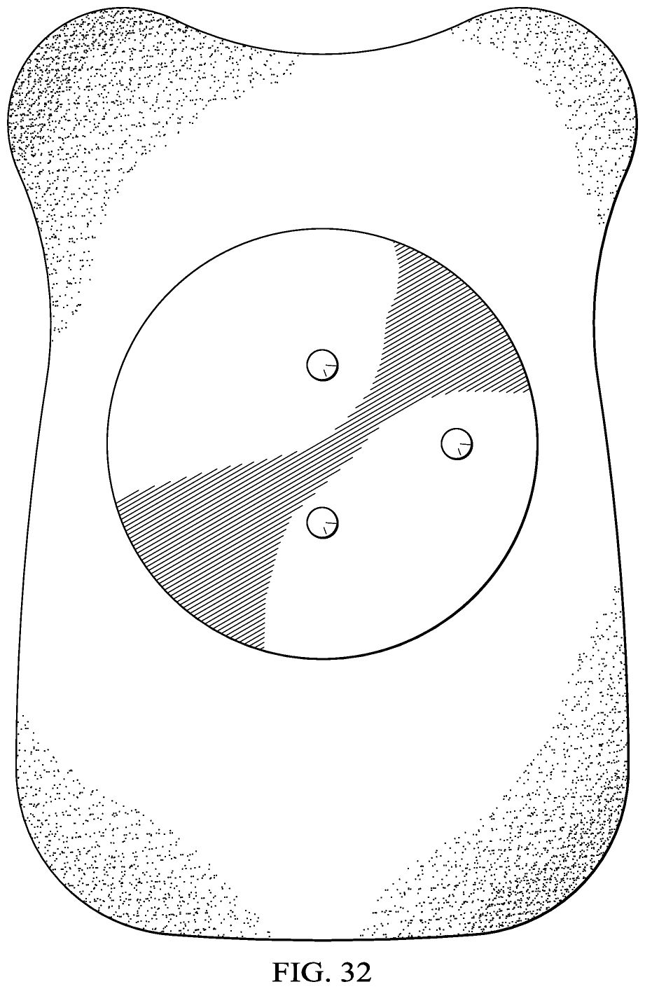

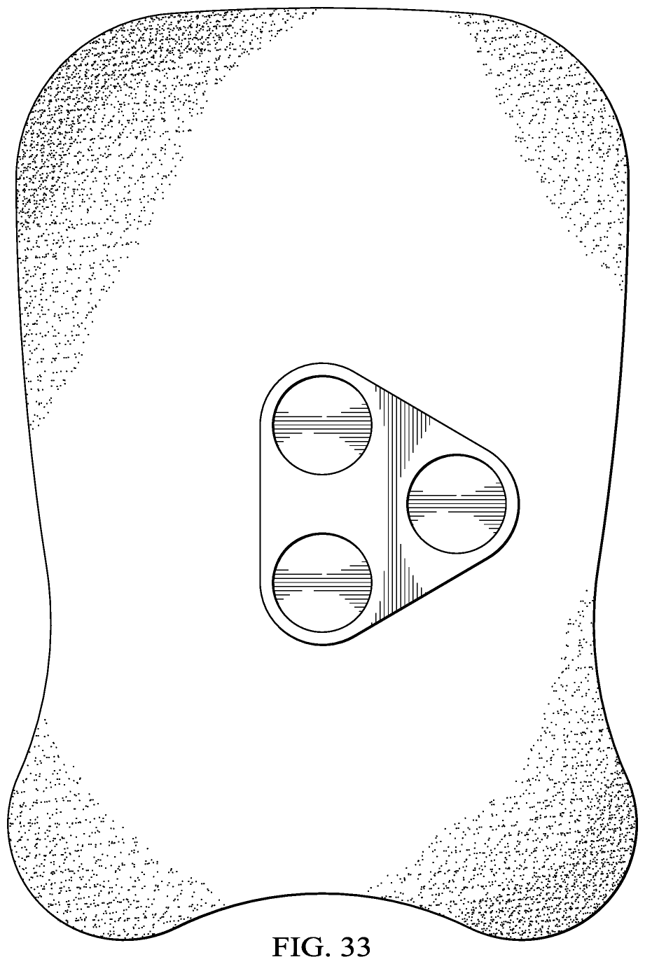

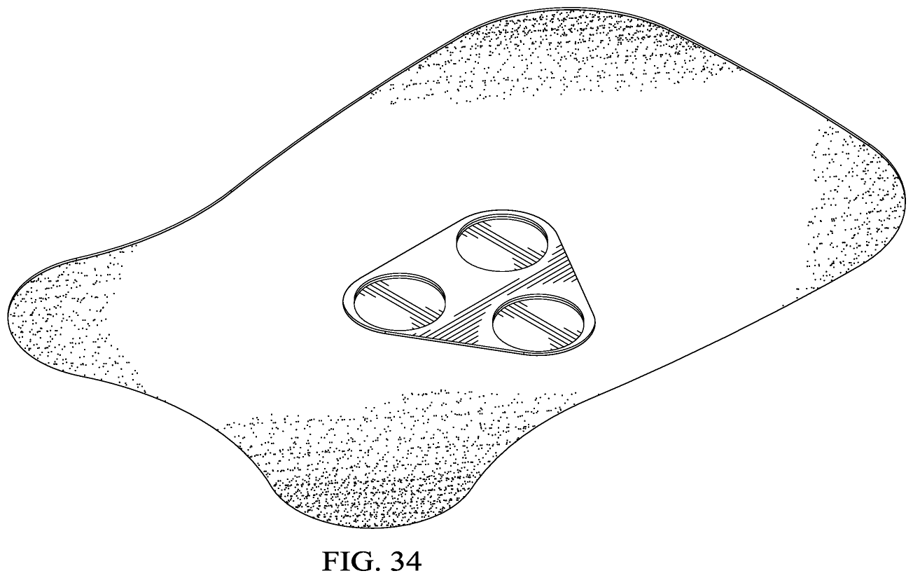

FIGS. 27-34 illustrate the embodiment of FIG. 11, but without a tab 16.

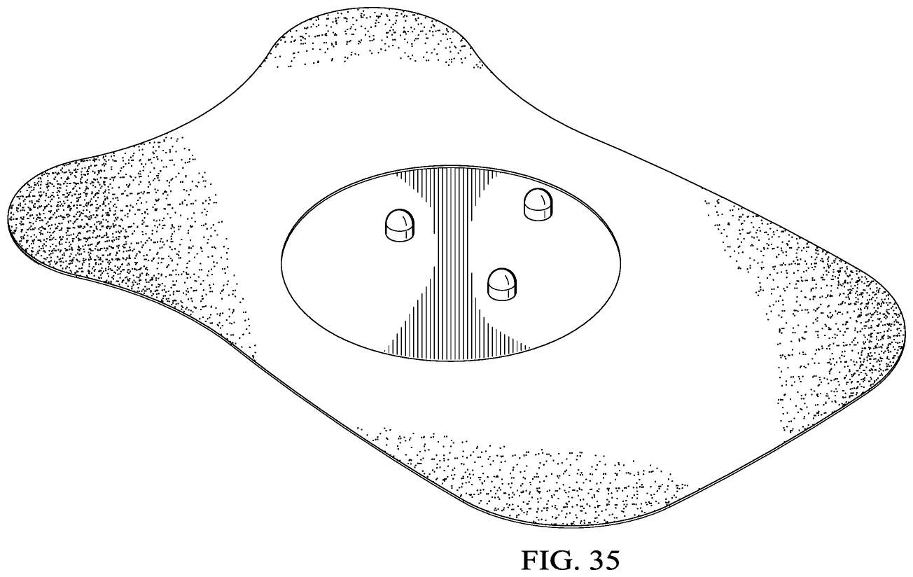

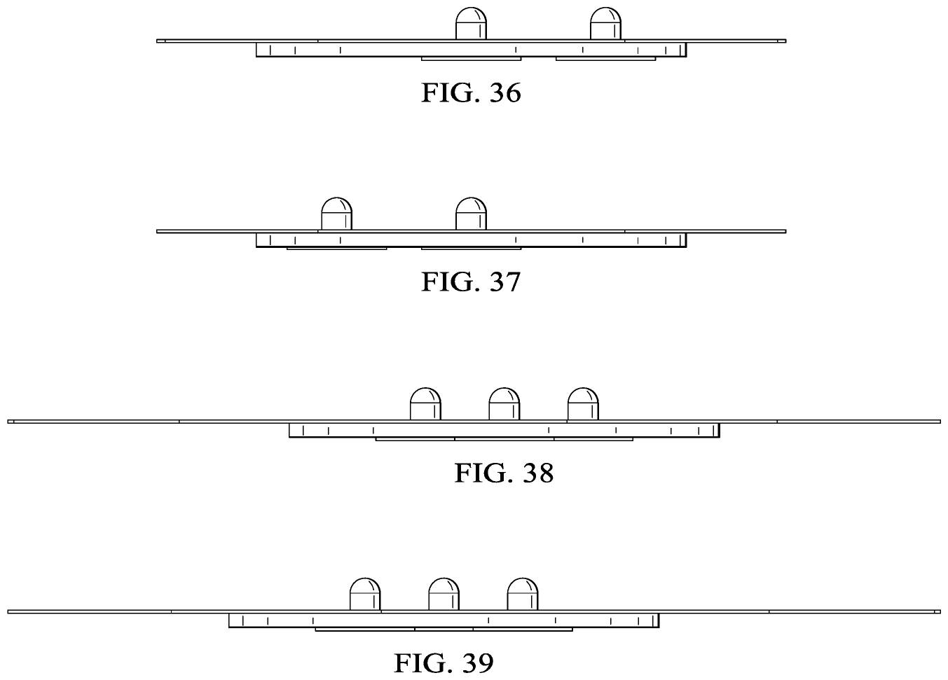

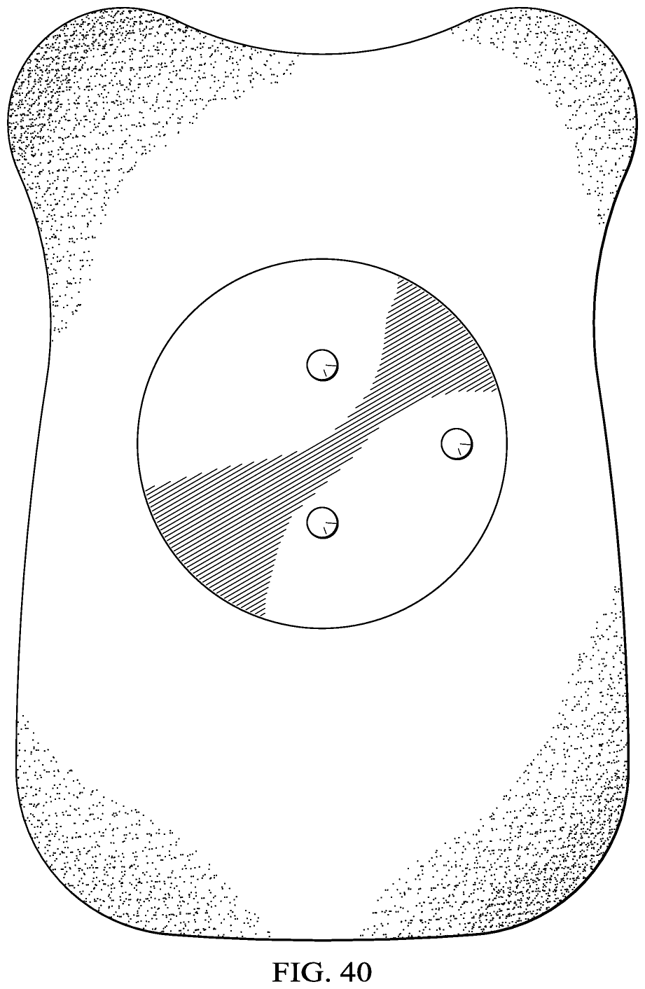

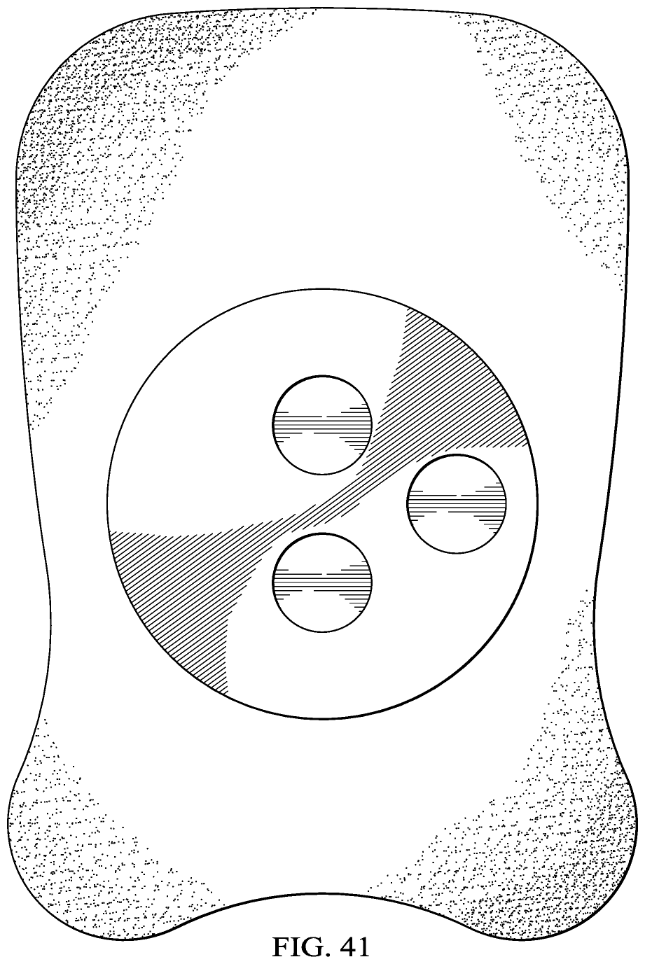

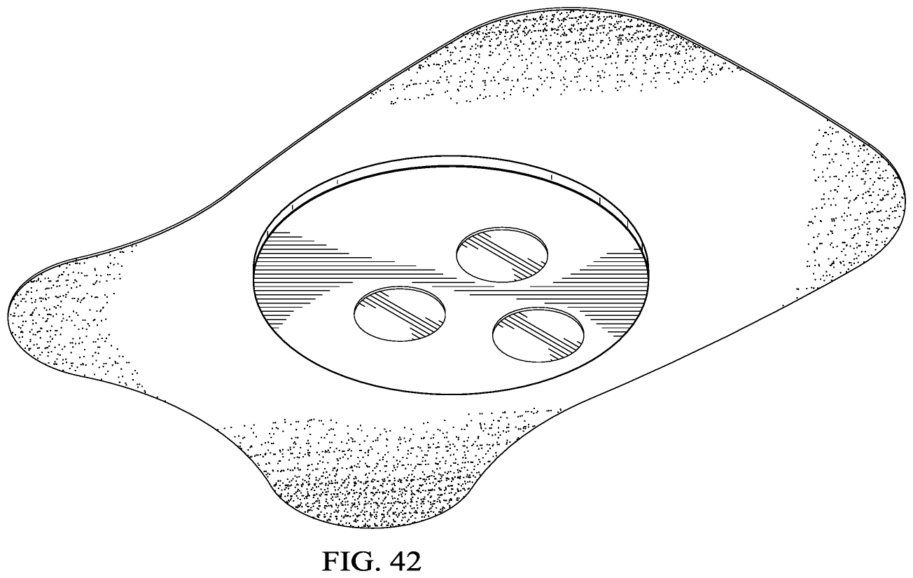

FIGS. 35-42 illustrate the embodiment of FIG. 19, but without a tab 214.

In yet other embodiments, the patch body and electrode base may comprise a single item of manufacture comprising a more flexible portion as patch body and a less flexible portion as electrode base. In other embodiments, an electrode base may be disposed between and at least partially enclosed by two flexible layers that together form a patch body. A patch body may comprise a plurality of apertures, each aperture being configured to allow exposure of one electrode stud or hydrogel cap. The electrode studs may comprise snap-like bodies configured to snap into electrode receptacles on a sensor device.

In some embodiments, the sensor device may comprise a processor, transceiver and power supply. The sensor device may receive electromyography signals from the human body through the hydrogel caps and electrode studs. The device may process the signals, such as for epileptic seizure detection, and may transmit the signals and/or alerts to a caregiver. The electrode patch may be configured to carry the weight of the sensor device for several hours during a range of human activity, such as sleeping, physical exercise, and personal hygiene.

An electrode patch may be thus variously embodied and formed, such as described in the following clauses:

1. An electrode patch comprising: a patch body having a skin side and a device side, the skin side having an adhesive coating; an electrode base coupled to the patch body; a plurality of electrode studs securely mounted to the electrode body, the electrode studs each being configured for electrical connection to a sensor device at the device side of the patch body; and a plurality of hydrogel caps securely mounted to the electrode studs, each of the plurality of hydrogel caps being in electrical contact with one of the plurality of electrode studs.

2. The electrode patch of clause 1, wherein the electrode base is coupled to the skin side of the patch body.

3. The electrode patch of clause 2, wherein the patch body forms an aperture through which the electrode studs may protrude.

4. The electrode patch of clause 3, wherein the electrode base is coupled to the device side of the patch body.

5. The electrode patch of clause 4, wherein the patch body forms an aperture through which the hydrogel caps may protrude.

6. The electrode patch of clauses 1, 2, 3, 4 or 5, further comprising a release tab affixed to the patch body.

7. The electrode patch of clause 6, wherein the release tab is removably affixed to the patch body.

8. The electrode patch of clause 6, wherein the release tab is permanently affixed to the patch body.

9. The electrode patch of clauses 7 or 8, wherein the release tab is affixed to the skin side of the patch body.

10. The electrode patch of clauses 7 or 8, wherein the release tab is affixed to the device side of the patch body.

11. The electrode patch of clause 1 further comprising a release liner removably affixed to the adhesive coating.

12. The electrode patch of clauses 7 or 8, wherein the release tab comprises a stiffer material than the patch body.

13. The electrode patch of clause 1 further comprising a plurality of lobes configured to reduce patch-skin shear forces along a plurality of force vectors.

14. The electrode patch of clause 1 further comprising a flexible material.

15. The electrode patch of clause 14, the flexible material comprising one of a fabric, a mesh, a breathable membrane, or a plastic.

16. The electrode patch of clause 1, the electrode studs each comprising a conductive material.

17. The electrode patch of clause 16, the electrode studs each comprising metal.

18. The electrode patch of clause 1, the electrode studs each comprising a non-conductive material having a coating comprising a conductive material.

19. The electrode patch of clause 1, wherein the non-conductive material is one of a plastic, ceramic or glass, and the coating is metal.

20. The electrode patch of clause 1, the electrode studs each having a snap-like body configured for removable reception by a sensor device.

21. The electrode patch of clause 1, the electrode studs each comprising a wide base, and each hydrogel cap being affixed to a wide base of an electrode stud.

22. The electrode patch of clause 1, each electrode stud being mounted to the electrode body so as to form a shallow depression in which a hydrogel cap may be disposed.

23. The electrode patch of clause 22, further comprising a spacer disposed between the electrode body and the patch body, the spacer forming an aperture well for each hydrogel cap.

24. The electrode patch of clause 23, wherein the spacer and electrode body are permanently joined.

25. The electrode patch of clause 23, wherein the spacer and electrode body form a unitary item of manufacture.

26. The electrode patch of clause 24, wherein the patch body is configured for removably mounting over a biceps muscle.

27. The electrode patch of clause 1, the patch body being a first patch body, the electrode patch further comprising a second patch body, the electrode body being disposed between the first patch body and the second patch body.

28. The electrode patch of clause 27, the electrode body being at least partially enclosed by the first patch body and the second patch body.

29. The electrode patch of clause 1, the patch body and electrode body comprising a unitary item of manufacture, the patch body forming a more flexible portion and the electrode body forming a more rigid portion.

30. The electrode patch of clause 1, wherein the adhesive coating comprises a pressure-sensitive adhesive.

31. The electrode patch of clause 1, each of the plurality of hydrogel caps comprising a gel of sufficient elasticity to substantially return to its shape after deformation by application to a human body.

32. The electrode patch of clause 1, each of the plurality of hydrogel caps comprising a gel of sufficient viscosity that removal of the caps from the human body will leave substantially no residue.

33. The electrode patch of clause 1, each of the plurality of hydrogel caps comprising a gel of sufficient viscosity that removal of the caps from the human body will leave insufficient residue to short circuit two of the plurality of electrodes.

34. The electrode patch of clause 1, the plurality of electrode studs being three.

Although the disclosed subject matter and its advantages have been described in detail, it should be understood that various changes, substitutions and alterations can be made herein without departing from the invention as defined by the appended claims. Moreover, the scope of the claimed subject matter is not intended to be limited to the particular embodiments of the process, machine, manufacture, composition, or matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods or steps.

We claim:

1. An electrode patch comprising:
a patch body having a skin side and a device side, the skin side having an adhesive coating;
an electrode base coupled to the patch body;
a plurality of electrode studs securely mounted to the electrode body, the electrode studs each being configured for electrical connection to a sensor device at the device side of the patch body; and
a plurality of hydrogel caps securely mounted to the electrode studs, each of the plurality of hydrogel caps being in electrical contact with one of the plurality of electrode studs.

2. The electrode patch of claim 1, wherein the electrode base is coupled to the skin side of the patch body.

3. The electrode patch of claim 2, wherein the patch body forms an aperture through which the electrode studs may protrude.

4. The electrode patch of claim 3, wherein the electrode base is coupled to the device side of the patch body.

5. The electrode patch of claim 4, wherein the patch body forms an aperture through which the hydrogel caps may protrude.

6. The electrode patch of claim 1, 2, 3 4 or 5, further comprising a release tab affixed to the patch body.

7. The electrode patch of claim 6, wherein the release tab is removably affixed to the patch body.

8. The electrode patch of claim 6, wherein the release tab is permanently affixed to the patch body.

9. The electrode patch of claim 7 or 8, wherein the release tab is affixed to the skin side of the patch body.

10. The electrode patch of claim 7 or 8, wherein the release tab is affixed to the device side of the patch body.

11. The electrode patch of claim 1 further comprising a release liner removably affixed to the adhesive coating.

12. The electrode patch of claim 7 or 8, wherein the release tab comprises a stiffer material than the patch body.

13. The electrode patch of claim 1 further comprising a plurality of lobes configured to reduce patch-skin shear forces along a plurality of force vectors.

14. The electrode patch of claim 1 further comprising a flexible material.

15. The electrode patch of claim 14, the flexible material comprising one of a fabric, a mesh, a breathable membrane, or a plastic.

16. The electrode patch of claim 1, the electrode studs each comprising a conductive material.

17. The electrode patch of claim 16, the electrode studs each comprising metal.

18. The electrode patch of claim 1, the electrode studs each comprising a non-conductive material having a coating comprising a conductive material.

19. The electrode patch of claim 1, wherein the non-conductive material is one of a plastic, ceramic or glass, and the coating is metal.

20. The electrode patch of claim 1, the electrode studs each having a snap-like body configured for removable reception by a sensor device.

21. The electrode patch of claim 1, the electrode studs each comprising a wide base, and each hydrogel cap being affixed to a wide base of an electrode stud.

22. The electrode patch of claim 1, each electrode stud being mounted to the electrode body so as to form a shallow depression in which a hydrogel cap may be disposed.

23. The electrode patch of claim 22 further comprising a spacer disposed between the electrode body and the patch body, the spacer forming an aperture well for each hydrogel cap.

24. The electrode patch of claim 23, wherein the spacer and electrode body are permanently joined.

25. The electrode patch of claim 23, wherein the spacer and electrode body form a unitary item of manufacture.

26. The electrode patch of claim 23, wherein the patch body is configured for removably mounting over a biceps muscle.

27. The electrode patch of claim 1 the patch body being a first patch body, the electrode patch further comprising a second patch body, the electrode body being disposed between the first patch body and the second patch body.

28. The electrode patch of claim 27, the electrode body being at least partially enclosed by the first patch body and the second patch body.

29. The electrode patch of claim 1, the patch body and electrode body comprising a unitary item of manufacture, the patch body forming a more flexible portion and the electrode body forming a more rigid portion.

30. The electrode patch of claim 1, wherein the adhesive coating comprises a pressure-sensitive adhesive.

31. The electrode patch of claim 1, each of the plurality of hydrogel caps comprising a gel of sufficient elasticity to substantially return to its shape after deformation by application to a human body.

32. The electrode patch of claim 1, each of the plurality of hydrogel caps comprising a gel of sufficient viscosity that removal of the caps from the human body will leave substantially no residue.

33. The electrode patch of claim 1, each of the plurality of hydrogel caps comprising a gel of sufficient viscosity that removal of the caps from the human body will leave insufficient residue to short circuit two of the plurality of electrodes.

34. The electrode patch of claim 1, the plurality of electrode studs being three.
